(19) United States
(12) Patent Application Publication
(10) Pub. No.: US 2019/0365562 A1
(43) Pub. Date: Dec. 5, 2019

(54) Prosthesis Heating Apparatus

(71) Applicant: David Kreher, (US)

(72) Inventor: Wesley Russell, Memphis, TN (US)

(73) Assignee: Wesley Russell, Memphis, TN (US)

(21) Appl. No.: 15/996,494

(22) Filed: Jun. 3, 2018

(51) Int. Cl. A61F 7/00; A61F 2/68

(52) U.S. Cl. CPC A61F 7/00; A61F 2002/7665; A61F 2/68

(57) ABSTRACT

A prosthesis heating apparatus for warming the entire prosthesis to prevent the prosthesis from withdrawing heat from the wearer's body, in particular, from withdrawing body heat from the bone to which the prosthesis is attached. The prosthetic heating apparatus can use cyclical timer switch or the disclosed dual switched circuit for turning an electric device on and off at set intervals.

### CROSS-REFERENCE TO RELATED APPLICATIONS

| | | |
|---|---|---|
| 15/596,493 | Jun. 3, 2018 | Russell |

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### FIELD OF THE INVENTION

The field of the invention presently disclosed includes a prosthetic heating apparatus the wraps the prosthetic with a sleeve comprising heating elements operated by battery power, which allows the wearer of the prosthetic to participate in cold weather activities and be exposed to cold weather without risking exposure of the limb to the cold temperatures down to the bone, where the prosthetic is attached to the limb.

### BACKGROUND OF THE INVENTION

Prosthetic devices are generally connected directly to the bone by means of a pin and are frequently made of a metallic material. During cold weather, as a conductor, the prosthetic can withdraw body heat from the wearer through the pin and into the prosthetic causing chilling of the body down to the bone. This chilling can prevent a wearer of a prosthetic from participating in cold weather events or being exposed cold weather for more than short periods of time. Rewarming of the limb of the wearer of the prosthetic can take an extended period of time because again, it is not just the body surface that is code, but warmth has been taken from the internal elements including the bone. Traditional prosthetic heating mechanisms operate only on the socket of the prosthetic and do not address the problem caused by heat being drawn through the pin and into the rest of the prosthetic. The present disclosure is a prosthetic heating apparatus that warms the entire prosthetic, rather than just the socket.

### SUMMARY OF THE INVENTION

The present disclosure reveals a prosthesis heating apparatus for warming the entire prosthesis of the wearer comprising a sleeve with hearing elements and a systems device. The systems device comprises the power storage device and the controllers to operate the prosthesis heating apparatus. The prosthesis heating apparatus is operated by a plurality of controllers that include such functions as a power supply as well as regulating when electricity is supplied to the heating elements and when it is not through either timing mechanisms or temperature parameters.

The switching mechanism can be operated by a cyclical timer switch common to the industry or through a revealed dual switched circuit for controlling how long current is supplied to an electrical device and how long current is prevented from flowing to an electrical device, wherein this sequence can be repeated, with a first switch and a second switch. The timer and switches could be operated mechanically or as an electronic circuit.

### BRIEF DESCRIPTION OF THE SEVERAL VIEWS OF THE DRAWING

FIG. 1 is a view of a prosthetic limb;

FIG. 2A is a view of the prosthesis heating apparatus with the system device attached by appropriate wiring;

FIG. 2B is a view of the prosthesis heating apparatus with the system device attached directly to the heating elements;

FIG. 3A is a schematic of the;

FIG. 3B is a schematic of the first embodiment of the dual control circuit;

FIG. 3C is a schematic of the second embodiment of the dual control circuit;

FIG. 3D is a schematic of the third embodiment of the dual control circuit

FIG. 4A is a schematic of the internal circuitry of the first switch; and

FIG. 4B is a schematic of the internal circuitry of the second switch.

FIG. 5A is a view of the first embodiment of the controllers of the system device with three controllers;

FIG. 5B is a view of the first embodiment of the controllers of the system device with four controllers;

FIG. 5C is a view of the second embodiment of the controllers of the system device wherein the first controller and the second controller are combined into a single controller;

FIG. 5D is a view of the third embodiment of the controllers of the system device wherein the second controller and the third controller are combined into a single controller;

FIG. 5E is a view of the fourth embodiment of the controllers of the system device wherein the first controller, the second controller, and third controller are combined into a single controller; and

FIG. 5F is a view of the fifth embodiment of the controllers of the system device wherein the first controller and the fourth controller are combined into a single controller and wherein the second and third controllers are automatic and not adjustable.

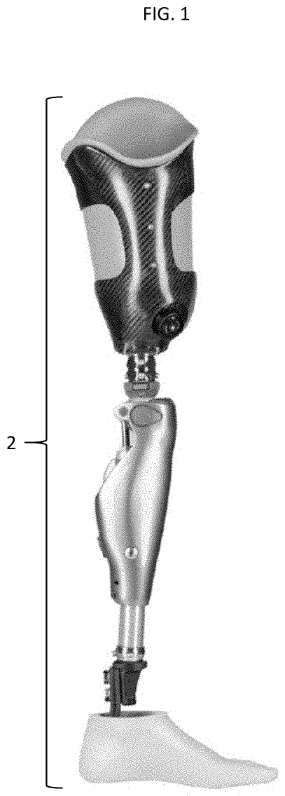

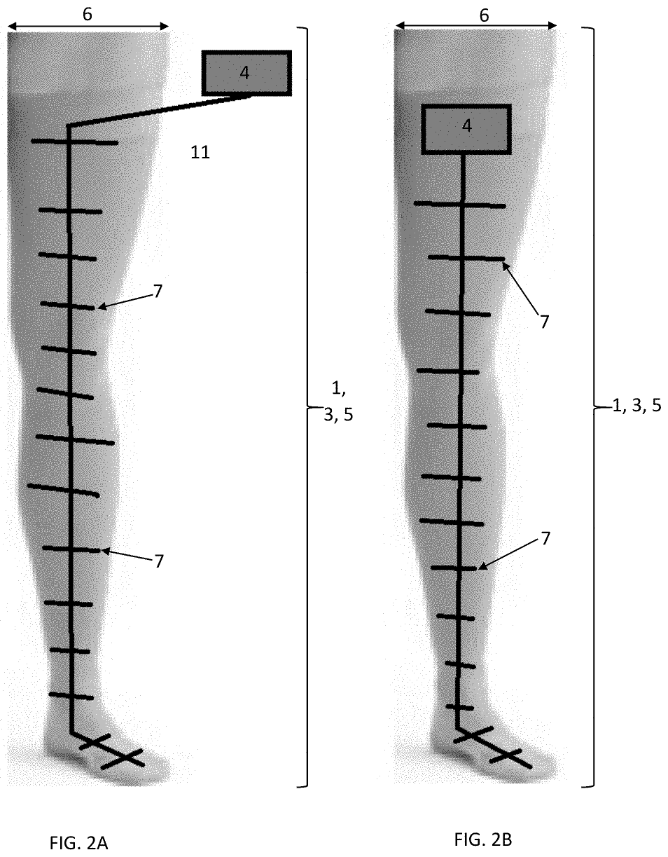

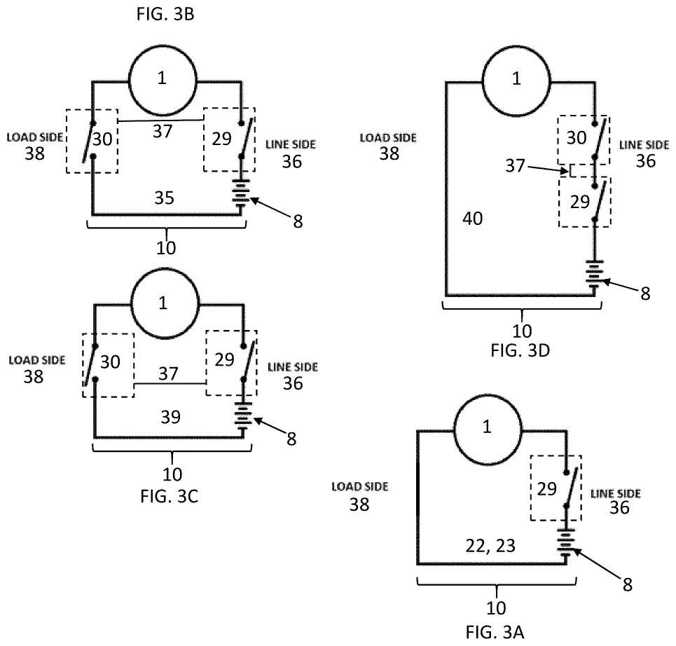

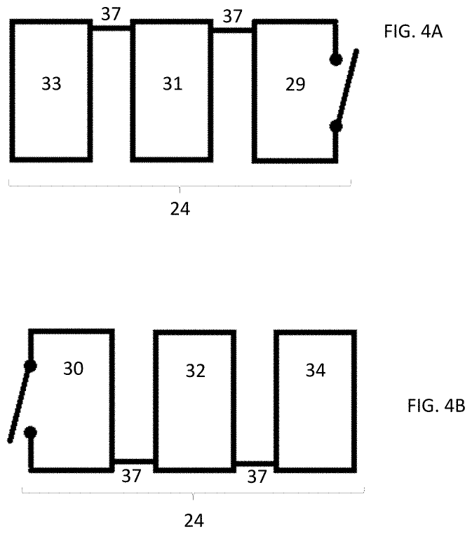

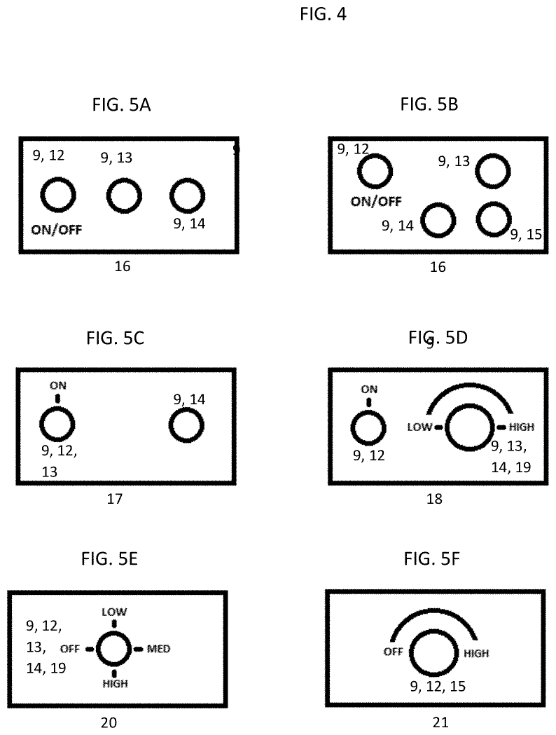

### DETAILED DESCRIPTION OF THE INVENTION

The present disclosure reveals a prosthetic heating apparatus 1 for warming all of a prosthetic device 2 while the wearer is exposed to cold temperatures, said prosthetic heating apparatus 1 comprising a sleeve 3 and a systems device 4 attached to the sleeve 3.

The sleeve 3 comprises a length 5 and circumference 6, being capable of sliding over and wrapping the entire prosthetic device 2, and further comprising a plurality of heating elements 7 along the length 5 and circumference 6 of the sleeve 3.

The systems device 4 comprises a power storage device 8, a plurality of controllers 9, wherein the plurality of controllers 9 is at least one controller 9, a switching mechanism 10, and appropriate electrical wiring 37. The power storage device 8 is capable of supplying electricity so that the heating elements 7 of the sleeve 3 can convert said electricity into heat and the power storage device 8 may be rechargeable. The switching mechanism 10 is capable of turning on and off the flow of electricity to the plurality of heating elements 7. The plurality of controllers 9 includes a first controller capable 12 of turning on the prosthetic heating apparatus 1, a second controller 13 capable of controlling the amount of time the switching mechanism 10 allows electricity to be sent to plurality of heating elements 7, and a third controller 14 capable of controlling the length of time between intervals wherein the switching mechanism 10 does not allow electricity to flow to the plurality of heating elements 7.

The power storage device 8 is connected to the switching mechanism 10, the plurality of controllers 9 are each connected to the switching mechanism 10 by appropriate electrical wiring 37, and the switching mechanism 10 is connected to the plurality of heating elements 7 of the sleeve 3. When the first controller 12 is switched on, the power storage device 8 supplies electricity to the switching mechanism 10 and the second controller 12 and the third controller 13 work together to determine when electricity is sent to the plurality of heating elements 7 of the sleeve 3 and when power is not being sent to the plurality of heating elements 7 of the sleeve 3.

The prosthetic heating apparatus 1 may also have a fourth controller 15 of the plurality of controllers 9 wherein said fourth controller 15 is capable of controlling the amount of electricity flowing from the power storage device 8 to the switching mechanism 10.

The plurality of controllers 9 can be combined in several embodiments. In a first embodiment 16 all of the controllers of the plurality of controllers 9 are stand-alone controllers. In the second embodiment 17 of the plurality of controllers 9, the first controller 12 and the second controller 13 are combined into a single controller. In the third embodiment 18 of the plurality of controllers 9 the second controller 13 and the third controller 14 are combined into a temperature sensor 19 wherein the temperature sensor 19 can be turned on and sent an appropriate temperature range such that, when the temperature sensor 19 detects a temperature below the input range electricity is supplied to the plurality of heating elements 7 of the sleeve 3 and when the temperature sensor 19 detects a temperature at the maximum of the desired range, electricity is stopped being delivered to the plurality of heating elements 7 of the sleeve 3. In this embodiment the appropriate temperature range may be designated by indicators such as low, medium and high or otherwise. In the fourth embodiment 20 of the plurality of controllers 9, the first controller 12, the second controller 13, and the third controller 14 are combined into a temperature sensor 19 wherein the temperature sensor 19 can be turned on and sent an appropriate temperature range such that, when the temperature sensor 19 detects a temperature below the input range electricity is supplied to the plurality of heating elements 7 of the sleeve 3 and when the temperature sensor 19 detects a temperature at the maximum of the desired range, electricity is stopped being delivered to the plurality of heating elements 7 of the sleeve 3. In this embodiment the appropriate temperature range may be designated by indicators such as low, medium and high or otherwise. The fifth embodiment 21 of the plurality of controllers 9 comprises the first controller 12 and the fourth controller 15 are combined into a single controller. In this embodiment, the second controller 13 and third controller 14 may be automatically preset or adjustable. These combinations are examples and do not restrict the possibility of other combinations of the plurality of controllers 9.

The switching mechanism 10 of the prosthetic heating apparatus 1 may also have several embodiments. The first embodiment 22 is a type common in the industry is a cyclical timer switch 23. The inventor also discloses a dual switched circuit 24 for the controlling of an electrical device on a timed on-off loop with three embodiments as disclosed in U.S. patent application Ser. No. 15/596,493.

In each of the embodiments of the dual switched circuit 24, the dual switched circuit comprises a first switch 29, a second switch 30, a first timer 31, a second timer 32, a first controller 33, and a second controller 34.

In the first embodiment 35 of the dual switched circuit 24, the first switch 29 is located on the line side 36 of the prosthetic heating apparatus 1, comprises the first timer 31 and the first controller 33. The first controller 33 is connected to the first timer 31 by appropriate electrical wiring 37 and the first timer 31 is attached to the first switch 29 by appropriate electrical wiring 37. The first switch 29 is capable of receiving a signal from the first timer 31 that indicates when the first switch 29 should open and wherein the first switch 29 is also able to convey that same signal to the second switch 30 by appropriate electrical wiring 37, whereupon the second switch 30 also opens. The first controller 33 is able to set the interval during which the first timer 31 allows the first switch 29 and the second switch 30 to remain closed and thus allow current to be supplied to the prosthetic heating apparatus 1. The second switch 30 is located on the load side 38 of the prosthetic heating apparatus 1, comprises the second timer 32 and the second controller 34. The second controller 34 is connected to the second timer 32 by appropriate electrical wiring 37 and the second timer 32 is attached to the second switch 30 by appropriate electrical wiring 37. The second switch 30 is capable of receiving a signal from the second timer 32 that indicates when the second switch 30 should close and wherein the second switch 30 is also able to convey that same signal to the first switch 29 by appropriate electrical wiring 37, whereupon the first switch 29 also closes. The second controller 34 being able to set the interval during which the second timer 32 allows the first switch 29 and the second switch 30 to remain open and thus prevent current from being supplied to the prosthetic heating apparatus 1.

In the second embodiment 39 of the dual switched circuit 24, the first switch 29 is located on the load side 38 of the prosthetic heating apparatus 1, comprises the first timer 31 and the first controller 33. The first controller 33 is connected to the first timer 31 by appropriate electrical wiring 37 and the first timer 31 is attached to the first switch 29 by appropriate electrical wiring 37. The first switch 29 is capable of receiving a signal from the first timer 31 that indicates when the first switch 29 should open and wherein the first switch 29 is also able to convey that same signal to the second switch 30 by appropriate electrical wiring 37, whereupon the second switch 30 also opens. The first controller 33 is able to set the interval during which the first timer 31 allows the first switch 29 and the second switch 30 to remain closed and thus allow current to be supplied to the prosthetic heating apparatus 1. The second switch 30 is located on the line side 36 of the prosthetic heating apparatus 1, comprises the second timer 32 and the second controller 34. The second controller 34 is connected to the second timer 32 by appropriate electrical wiring 37 and the second timer 32 is attached to the second switch 30 by appropriate electrical wiring 37. The second switch 30 is capable of receiving a signal from the second timer 32 that indicates when the second switch 30 should close and wherein the second switch 30 is also able to convey that same signal to the first switch 29 by appropriate electrical wiring 37, whereupon the first switch 29 also closes. The second controller 34 being able to set the interval during which the second timer 32 allows the first switch 29 and the second switch 30 to remain open and thus prevent current from being supplied to the electric device 2.

In the third embodiment 40 of the dual switched circuit 24, the first switch 29 is located on the line side 36 of the prosthetic heating apparatus 1, comprises the first timer 31 and the first controller 33. The first controller 33 is connected to the first timer 31 by appropriate electrical wiring 37 and the first timer 31 is attached to the first switch 29 by appropriate electrical wiring 37. The first switch 29 is capable of receiving a signal from the first timer 31 that indicates when the first switch 29 should open and wherein the first switch 29 is also able to convey that same signal to the second switch 30 by appropriate electrical wiring 37, whereupon the second switch 30 also opens. The first controller 33 is able to set the interval during which the first timer 31 allows the first switch 29 and the second switch 30 to remain closed and thus allow current to be supplied to the prosthetic heating apparatus 1. The second switch 30 is located on the line side 36 of the prosthetic heating apparatus 1, comprises the second timer 32 and the second controller 34. The second controller 34 is connected to the second timer 32 by appropriate electrical wiring 37 and the second timer 32 is attached to the second switch 30 by appropriate electrical wiring 37. The second switch 30 is capable of receiving a signal from the second timer 32 that indicates when the second switch 30 should close and wherein the second switch 30 is also able to convey that same signal to the first switch 29 by appropriate electrical wiring 37, whereupon the first switch 29 also closes. The second controller 34 being able to set the interval during which the second timer 32 allows the first switch 29 and the second switch 30 to remain open and thus prevent current from being supplied to the prosthetic heating apparatus 1.

In any embodiment of the dual switched circuit 24, the first timer 31 and the second timer 32 can be either mechanical timers or electronic timers.

What is claimed:

1. A prosthetic heating apparatus for warming all of a prosthetic device while the wearer is exposed to cold temperatures, said prosthetic heating apparatus comprising:
a sleeve and a systems device attached to the sleeve;
the sleeve comprising a length and circumference, being capable of sliding over and wrapping the entire prosthetic device, and further comprising a plurality of heating elements along the length and circumference of the sleeve;
the systems device comprising a power storage device, a plurality of controllers, a switching mechanism, and appropriate wiring;
wherein the power storage device is capable of supplying electricity so that the heating elements of the sleeve can convert said electricity into heat;
wherein the power storage device is at least one of rechargeable and/or replaceable wherein the switching mechanism is capable of turning on and off the flow of electricity to the heating elements;
wherein the plurality of controllers includes a first controller capable of turning on the prosthetic heating apparatus, a second controller capable of controlling the amount of time the switching mechanism allows electricity to be sent to heating elements, and a third controller capable of controlling the length of time between intervals wherein the switching mechanism does not allow electricity to flow to the heating element;
wherein, the power storage device is connected to the switching mechanism, the plurality of controllers are each connected to the switching mechanism by appropriate wiring, and the switching mechanism is connected to the heating elements of the sleeve; and
wherein, when the first controller is switched on, the power storage device supplies electricity to the switching mechanism and the second controller and the third controller work together to determine when electricity is sent to the heating elements of the sleeve and when power is not being sent to the heating elements of the sleeve.

2. The prosthetic heating apparatus of claim 1 wherein the first controller and the second controller are combined into a single controller.

3. The prosthetic heating apparatus of claim 1 wherein the second controller and the third controller are combined into a temperature sensor controller wherein the temperature sensor controller can be turned on and sent an appropriate temperature range such that, when the temperature sensor detects a temperature below the input range electricity is supplied to the heating elements of the sleeve and when the temperature sensor detects a temperature at the maximum of the desired range, electricity is stopped being delivered to the heating elements of the sleeve.

4. The prosthetic heating apparatus of claim 1 wherein the first controller, the second controller, and the third controller are combined into a temperature sensor controller wherein the temperature sensor controller can be turned on and sent an appropriate temperature range such that, when the temperature sensor detects a temperature below the input range electricity is supplied to the heating elements of the sleeve and when the temperature sensor detects a temperature at the maximum of the desired range, electricity is stopped being delivered to the heating elements of the sleeve.

5. The prosthetic heating apparatus of claim 1 wherein there is a fourth controller of the plurality of controllers wherein said fourth controller is capable of controlling the amount of electricity flowing from the power storage device to the switching mechanism.

6. The prosthetic heating apparatus of claim 5 wherein the first controller and the fourth controller are combined into a single controller.

7. The prosthetic heating apparatus of claim 1 wherein the switching mechanism further comprises a dual switched circuit for the controlling of a electrical device on a timed on-off loop comprising first switch, a second switch, a first timer, a second timer, the second controller, and the third controller;
the first switch is located on the line side of the electrical device, comprises the first timer and the second controller, and wherein the second controller is connected to the first timer by appropriate wiring and the first timer is attached to the first switch by appropriate wiring;
wherein the first switch is capable of receiving a signal from the first timer that indicates when the first switch should open and wherein the first switch is also able to convey that same signal to the second switch by appropriate wiring, whereupon the second switch also opens;
the second controller being able to set the interval during which the first timer allows the first switch and the second switch to remain closed and thus allow current to be supplied to the electric device;
the second switch is located on the load side of the electrical device, comprises the second timer and the third controller, and wherein the third controller is connected to the second timer by appropriate wiring and the second timer is attached to the second switch by appropriate wiring;
wherein the second switch is capable of receiving a signal from the second timer that indicates when the second switch should close and wherein the second switch is also able to convey that same signal to the first switch by appropriate wiring, whereupon the first switch also closes; and
the third controller being able to set the interval during which the second timer allows the first switch and the second switch to remain open and thus prevent current from being supplied to the electric device.

8. The prosthetic heating apparatus of claim 7 wherein the first controller and the third controller are combined into the same controller.

9. The prosthetic heating apparatus of claim 7 wherein the first timer is a mechanical timer and the second timer is a mechanical timer.

10. The prosthetic heating apparatus of claim 9 wherein the first controller and the third controller are combined into the same controller.

11. The prosthetic heating apparatus of claim 7 wherein the first timer is an electronic timer and the second timer is an electronic timer.

12. The prosthetic heating apparatus of claim 11 wherein the first controller and the third controller are combined into the same controller.

13. The prosthetic heating apparatus of claim 1 wherein the switching mechanism further comprises a dual switched circuit for the controlling of a electrical device on a timed on-off loop comprising first switch, a second switch, a first timer, a second timer, the second controller, and the third controller;
the first switch is located on the load side of the electrical device, comprises the first timer and the second controller, and wherein the second controller is connected to the first timer by appropriate wiring and the first timer is attached to the first switch by appropriate wiring;
wherein the first switch is capable of receiving a signal from the first timer that indicates when the first switch should open and wherein the first switch is also able to convey that same signal to the second switch by appropriate wiring, whereupon the second switch also opens;
the second controller being able to set the interval during which the first timer allows the first switch and the second switch to remain closed and thus allow current to be supplied to the electric device;
the second switch is located on the line side of the electrical device, comprises the second timer and the third controller, and wherein the third controller is connected to the second timer by appropriate wiring and the second timer is attached to the second switch by appropriate wiring;
wherein the second switch is capable of receiving a signal from the second timer that indicates when the second switch should close and wherein the second switch is also able to convey that same signal to the first switch by appropriate wiring, whereupon the first switch also closes; and
the third controller being able to set the interval during which the second timer allows the first switch and the second switch to remain open and thus prevent current from being supplied to the electric device.

14. The prosthetic heating apparatus of claim 13 wherein the first controller and the third controller are combined into the same controller.

15. The prosthetic heating apparatus of claim 13 wherein the first timer is a mechanical timer and the second timer is a mechanical timer.

16. The prosthetic heating apparatus of claim 15 wherein the first controller and the third controller are combined into the same controller.

17. The prosthetic heating apparatus of claim 13 wherein the first timer is an electronic timer and the second timer is an electronic timer.

18. The prosthetic heating apparatus of claim 17 wherein the first controller and the third controller are combined into the same controller.

19. The prosthetic heating apparatus of claim 1 wherein the switching mechanism further comprises a dual switched circuit for the controlling of a electrical device on a timed on-off loop comprising first switch, a second switch, a first timer, a second timer, the second controller, and the third controller;
the first switch is located on the line side of the electrical device, comprises the first timer and the second controller, and wherein the second controller is connected to the first timer by appropriate wiring and the first timer is attached to the first switch by appropriate wiring;
wherein the first switch is capable of receiving a signal from the first timer that indicates when the first switch should open and wherein the first switch is also able to convey that same signal to the second switch by appropriate wiring, whereupon the second switch also opens;
the second controller being able to set the interval during which the first timer allows the first switch and the second switch to remain closed and thus allow current to be supplied to the electric device;
the second switch is located on the line side of the electrical device, comprises the second timer and the third controller, and wherein the third controller is connected to the second timer by appropriate wiring and the second timer is attached to the second switch by appropriate wiring;
wherein the second switch is capable of receiving a signal from the second timer that indicates when the second switch should close and wherein the second switch is also able to convey that same signal to the first switch by appropriate wiring, whereupon the first switch also closes; and
the third controller being able to set the interval during which the second timer allows the first switch and the second switch to remain open and thus prevent current from being supplied to the electric device.

20. The prosthetic heating apparatus of claim 19 wherein the first controller and the third controller are combined into the same controller.

21. The prosthetic heating apparatus of claim 19 wherein the first timer is a mechanical timer and the second timer is a mechanical timer.

22. The prosthetic heating apparatus of claim 21 wherein the first controller and the third controller are combined into the same controller.

23. The prosthetic heating apparatus of claim 19 wherein the first timer is an electronic timer and the second timer is an electronic timer.

24. The prosthetic heating apparatus of claim 23 wherein the first controller and the third controller are combined into the same controller.
